(19) United States
(12) Patent Application Publication
(10) Pub. No.: US 2025/0136916 A1
(43) Pub. Date: May 1, 2025

(54) CELL PROCESSING METHOD, CELL PROCESSING SYSTEM, CELL PROCESSING APPARATUS, AND STORAGE MEDIUM

(71) Applicant: CANON KABUSHIKI KAISHA, Tokyo (JP)

(72) Inventors: TAKUJI OKAMOTO, Tokyo (JP); TAKAYUKI TSUKIMOTO, Kanagawa (JP); NORIHIRO SUZUKI, Kanagawa (JP)

(21) Appl. No.: 18/929,382

(22) Filed: Oct. 28, 2024

(30) Foreign Application Priority Data
Oct. 30, 2023 (JP) 2023-186021

(51) Int. Cl. C12M 1/26; C12M 1/36

(52) U.S. Cl. CPC C12M 33/08; C12M 41/48

(57) ABSTRACT

A cell processing method for detaching cells adhering to a culture face of a culture container from the culture container by using a cell processing system including a vibration unit configured to generate vibrations includes heating a vibration transmission material to increase a temperature of the vibration transmission material by making the vibration unit generate a vibration in a first mode in a state where the vibration unit makes contact with the vibration transmission material, and detaching the cells from the culture container by making the vibration unit generate a vibration in a second mode different from the first mode after the heating is started, in a state where the vibration transmission material makes contact with the culture container.

### BACKGROUND

### Field of the Disclosure

The present disclosure relates to a cell processing method, a cell processing system, a cell processing apparatus, and a storage medium.

### Description of the Related Art

Cells produced by cell culturing have been used in the fields of drug development and regenerative medicine. In particular, in a case where a large amount of adherent cells are to be cultured, after the adherent cells adhering to a culture container and a scaffold material are detached and collected from the culture container and the scaffold material, the adherent cells are moved to another culture container and cultured again through a subculture operation. Further, even in a case where the subculture operation is not executed, it is desirable to detach the adherent cells from a culture container and a scaffold material when the adherent cells are to be used. Because a range of temperature at which the cells can have a high proliferation rate and/or a high survival rate varies depending on a cell type, a cell culturing process is generally executed by using an apparatus such as an incubator which is maintained at a constant temperature. However, when an operation such as a cell detaching operation is to be executed, a culture container has to be taken out from the incubator. Therefore, there is a possibility that a survival rate of cells is lowered because a temperature of the cells within the culture container placed outside the incubator becomes close to a room temperature.

According to a technique discussed in Japanese Patent Application Laid-Open No. 2005-328726, a heater is arranged on a placement table on which a culture container taken out from an incubation room is placed, so that a culture processing apparatus can maintain a culture medium within the culture container at a constant temperature.

### SUMMARY

Some embodiments of the present disclosure are directed to providing a cell processing method capable of adjusting a temperature of a member which makes contact with a culture container to a desirable range when cells adhering to a culture face of the culture container are to be detached. Further, some embodiments of the present disclosure are directed to providing a cell processing system and a cell processing apparatus capable of adjusting a temperature of a member which makes contact with a culture container to a desirable range when cells adhering to a culture face of the culture container are to be detached.

The above can be achieved by the present disclosure described below. That is, according to an aspect of the present disclosure, a cell processing method for detaching cells adhering to a culture face of a culture container from the culture container by using a cell processing system, including a vibration unit configured to generate vibrations, includes heating a vibration transmission material to increase a temperature of the vibration transmission material by making the vibration unit generate a vibration in a first mode in a state where the vibration unit makes contact with the vibration transmission material, and detaching the cells from the culture container by making the vibration unit generate a vibration in a second mode different from the first mode after the heating is started, in a state where the vibration transmission material makes contact with the culture container.

According to another aspect of the present disclosure, a cell processing apparatus that detaches cells adhering to a culture face of a culture container from the culture container includes a vibration unit configured to generate vibrations, and a control unit configured to control the vibrations generated by the vibration unit, wherein the control unit causes the vibration to be transmitted to a vibration transmission material to cause a temperature of the vibration transmission material to be increased by controlling the vibration unit to vibrate in a first mode in a state where the vibration unit makes contact with the vibration transmission material, and wherein the control unit causes the cells to be detached from the culture container by controlling the vibration unit to vibrate in a second mode different from the first mode in a state where the vibration transmission material makes contact with the culture container.

According to yet another aspect of the present disclosure, a cell processing system that detaches cells adhering to a culture face of a culture container from the culture container includes a vibration unit configured to generate vibrations, and a control unit configured to control the vibrations generated by the vibration unit, wherein the control unit causes the vibration to be transmitted to a vibration transmission material to cause a temperature of the vibration transmission material to be increased by controlling the vibration unit to vibrate in a first mode in a state where the vibration unit makes contact with the vibration transmission material, and wherein the control unit causes the cells to be detached from the culture container by controlling the vibration unit to vibrate in a second mode different from the first mode in a state where the vibration transmission material makes contact with the culture container.

Further features of various embodiments will become apparent from the following description of exemplary embodiments with reference to the attached drawings.

### BRIEF DESCRIPTION OF THE DRAWINGS

FIG. 1 is a schematic diagram illustrating an apparatus configuration of a cell processing system according to a first exemplary embodiment.

FIG. 2 is a flowchart illustrating a cell processing method according to the first exemplary embodiment.

FIG. 3 is a illustrative diagram of the cell processing method according to the first exemplary embodiment.

FIGS. 4A and 4B are schematic cross-sectional diagrams illustrating the cell processing system according to the first exemplary embodiment.

FIG. 5 is a conceptual diagram illustrating generation of heat according to the first exemplary embodiment.

FIGS. 6A and 6B are schematic diagrams illustrating a vibration mode according to the first exemplary embodiment.

FIG. 7 is a schematic diagram illustrating a temperature change according to the first exemplary embodiment.

FIG. 8 is a schematic diagram illustrating a local vibration according to a second exemplary embodiment.

FIGS. 9A to 9C are schematic diagrams illustrating vibration modes according to the second exemplary embodiment.

FIGS. 10A and 10B are schematic diagrams illustrating an example of changing the vibration mode according to the second exemplary embodiment.

FIG. 11 is a schematic diagram illustrating a configuration example of a cell processing apparatus according to a third exemplary embodiment.

FIG. 12 is a schematic diagram illustrating an apparatus configuration of a cell processing system according to the third exemplary embodiment.

FIG. 13 is a flowchart illustrating a cell processing method according to the third exemplary embodiment.

FIG. 14 is a flowchart illustrating a cell processing method according to the first exemplary embodiment.

### DESCRIPTION OF THE EMBODIMENTS

A type of heater and a heat transmission path from the heater to a culture container are not discussed in Japanese Patent Application Laid-Open No. 2005-328726. If the culture container is simply placed on a placement table having a heater, it will take time to transmit heat from the heater to the culture container. Therefore, there is a possibility that a temperature of the cells cannot be maintained sufficiently.

Inventors of the present disclosure conducted a study on a method for maintaining a temperature of the cells when the cells are detached from the culture container. As a result, it was found that, if a commonly-used heater is simply arranged inside a housing of the cell processing apparatus, a temperature of a vibration transmission material on which the culture container is placed may not be sufficiently increased because it will take time to increase a temperature of the heater.

The inventors further conducted a study based on the above knowledge, and found that the vibration transmission material can be heated by applying a vibration to the vibration transmission material before a culture container is placed. It was also found that, a temperature of the vibration transmission material can be increased more promptly by directly heating the vibration transmission material with the vibration of the vibration transmission material, than by only driving the heater.

Hereinafter, the present disclosure describes exemplary embodiments in further detail.

In the cell processing method according to a present exemplary embodiment, cells adhering to a culture face of the culture container are detached by transmitting a vibration generated by a vibration unit included in a cell processing system for generating a vibration to the culture container via a vibration transmission material. Further, in the cell processing method according to the present exemplary embodiment, in order to adjust a temperature of the cells contained in the culture container, the vibration unit is previously vibrated in a mode for increasing a temperature of the vibration transmission material, before the culture container makes contact with the vibration transmission material. In this way, a temperature of the cells contained in the culture container can be managed when the culture container is moved to the cell processing system from a culture environment such as an incubator.

### Cell Processing System

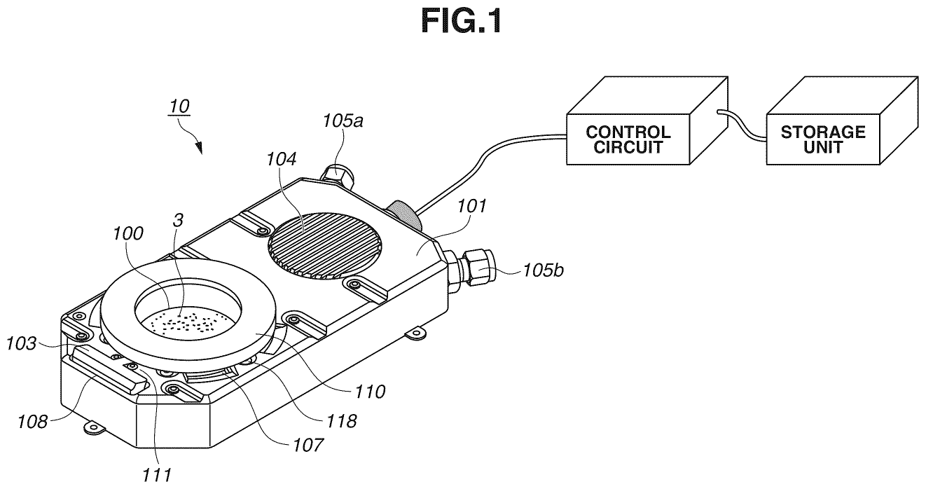

FIG. 1 is a schematic diagram illustrating an apparatus configuration of a cell processing system according to the present exemplary embodiment. The cell processing system according to the present exemplary embodiment includes at least a vibration unit for generating a vibration and a control unit for controlling the vibration generated by the vibration unit. A cell processing system 10 detaches cells 3 adhering to the culture face of a culture container 100 from the culture face by transmitting a vibration generated by the vibration unit to the cells 3 via a vibration transmission material (not illustrated). The culture container 100 is placed and held down by the weight of a dish weight 110 during the cell detachment process. A control circuit illustrated in FIG. 1 is one example of the control unit. The control circuit controls the vibration generated by the vibration unit. The control unit may include a module executed by a central processing unit (CPU) or a micro processing unit (MPU), or may include a circuit such as an application specific integrated circuit (ASIC) which executes a specific function. A temperature sensor 118 is disposed in nip contact with a vibration tub 107. The temperature sensor 118 is one example of a measuring unit. The temperature sensor 118 may be fixed with any fixing method such as bonding. This configuration enables direct or indirect temperature measurement for the culture surface of the culture container 100. Water tubes 105a and 105b are disposed for cooling so as to surround the vibration tub 107 for the purpose of temperature control. For example, the side of a water tube 105a is set as the inflow side of water (e.g., refrigerant), and the side of a water tube 105b is set as the outflow side of water. A storage tub 108 is disposed next to the vibration tub 107 in a groove form so that the vibration transmission fluid is freely movable between the two tubs through a fluid path 111. A sinking weight 103 is provided to allow the vibration transmission fluid to move from the vibration tub 107 to the storage tub 108 and vice versa through the fluid path 111.

In addition, some embodiments may be realized by a cell processing system to which a plurality of devices and arithmetic apparatuses are connected, and a cell processing apparatus configured of a single apparatus for executing a similar function is also included in the present disclosure.

### Vibration Unit

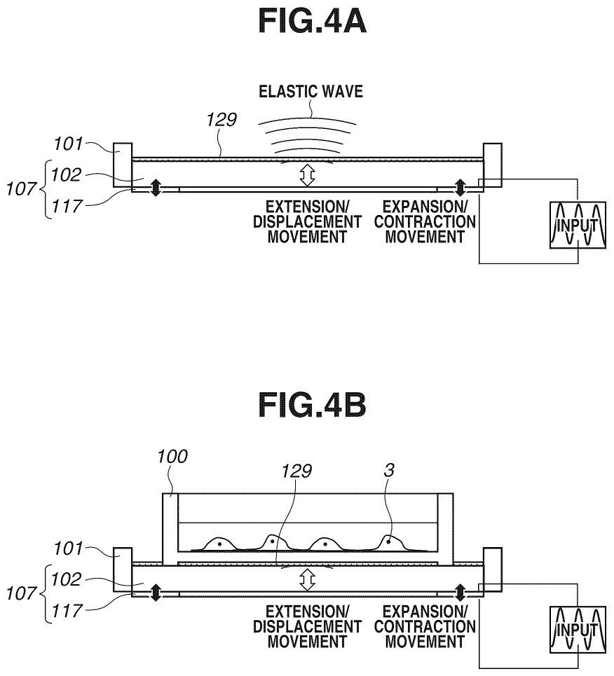

FIGS. 4A and 4B are schematic cross-sectional diagrams illustrating a cell processing system according to the present exemplary embodiment. FIG. 4A illustrates a state before the culture container 100 makes contact with the cell processing system. A vibration unit 107 illustrated in each of FIGS. 4A and 4B is a piezoelectric-type vibration unit. The vibration unit 107 includes a vibration plate 102 and a vibrator 117 adhering to each other. The vibrator 117 expands and contracts to vibrate when a driving voltage is applied to a piezoelectric material included in the vibrator 117. When the vibrator 117 expands and contracts to vibrate, the vibration plate 102 makes an extension/displacement movement, i.e., a flection/deformation movement. As a result, the vibration unit 107 vibrates to cause an elastic wave to be generated.

The vibration unit 107 can be a device such as a piezoelectric device or a motor capable of generating vibrations. In particular, in order to detach cells from the culture container 100, it is preferable that a device capable of generating a vibration of an ultrasonic band be used as the vibration unit 107. The vibrator 117 used for the vibration unit 107 can be a piezoelectric type or an electromagnetic type, as long as a vibration of a constant frequency can be generated thereby. In order to precisely control a vibration waveform and a vibration frequency, the vibrator 117 may be a piezoelectric-type vibrator. The piezoelectric type vibrator can be selected from a unimorph type, a bimorph type, and a bolt-clamped Langevin type.

### Housing

The cell processing system further includes a housing 101 for holding the vibration unit 107. As illustrated in FIGS. 4A and 4B, the housing 101 may have a bank-like projection on the upper side of the vibration unit 107 in order to hold the vibration transmission material when the cell processing system is placed on an experiment table. Further, a heater for increasing the temperature of the entire housing 101 and maintaining an equilibrium state thereof may be arranged inside the housing 101. By temperature management by both of the heater for maintaining a temperature of the entire housing 101 and the vibration unit 107 for increasing a temperature of the vibration transmission material, a temperature can promptly and efficiently be adjusted when the cell processing is to be executed.

### Vibration Transmission Material

When the cell processing system is to be used, a vibration transmission material 129 is placed to make contact with the vibration unit 107. The vibration transmission material 129 can be a material capable of transmitting at least part of the vibration generated by the vibration unit 107 to the culture container 100 from the vibration unit 107. In terms of a transmission capability, it may be preferable that the vibration transmission material 129 be a liquid or a solid. It may be much preferable that the vibration transmission material 129 contains water or silicone rubber. It is possible to effectively apply vibrations to the cells by making the vibration unit 107 vibrate in a state where the vibration transmission material 129 makes contact with both of the vibration unit 107 and the culture container 100.

### Culture Container

Any container that can be used to culture cells can be used as the culture container 100 described in the present exemplary embodiment. Further, a culture container internally storing a scaffold material such as a microcarrier or a hollow fiber material may also be used as the culture container 100. A dish, a flask, a well plate, and a culture bag are examples of the culture container 100.

A material capable of transmitting a vibration or a material capable of being deformed according to a vibration can be used as a material of the culture container 100. For example, the culture container 100 may be made of a polymer material such as polystyrene or a metallic material. The culture container 100 is placed to make contact with the vibration transmission material 129 placed on the vibration unit 107. In order to efficiently execute cell detachment, it is preferable that a face where the vibration transmission material 129 makes contact with the culture container 100 be located on the outer side of the culture container 100 when viewed from the culture face which the cells within the culture container 100 adhere to.

### Cells

The cell processing method according to the present exemplary embodiment can be applied to various types of cells. For example, the various types of cells include Chinese hamster ovary-derived cells (CHO cells), mouse connective tissue L929 cells, mouse skeletal muscle myoblast cells (C2C12 cells), human fetal lung-derived normal diploid fibroblast cells (TIG-3 cells), human embryonic kidney-derived cells (HEK 293 cells), human alveolar basal epithelial adenocarcinoma-derived cells (A 549 cells), human cervical cancer-derived cells (HeLa cells), epithelial cells, endothelial cells, skeletal muscle cells, smooth muscle cells, cardiac muscle cells, neuron cells, glial cells, fibroblast cells, hepatic parenchymal cells, hepatic nonparenchymal cells, fat cells, induced pluripotent stem (iPS) cells, embryonic stem (ES) cells, embryonic germ (EG) cells, embryonic carcinoma (EC) cells, mesenchymal stem cells, liver stem cells, pancreatic stem cells, skin stem cells, muscle stem cells, germ stem cells, progenitor cells of various tissues, and at least one selected from a group of cells differentiated and induced from these cells.

### Vibration Mode

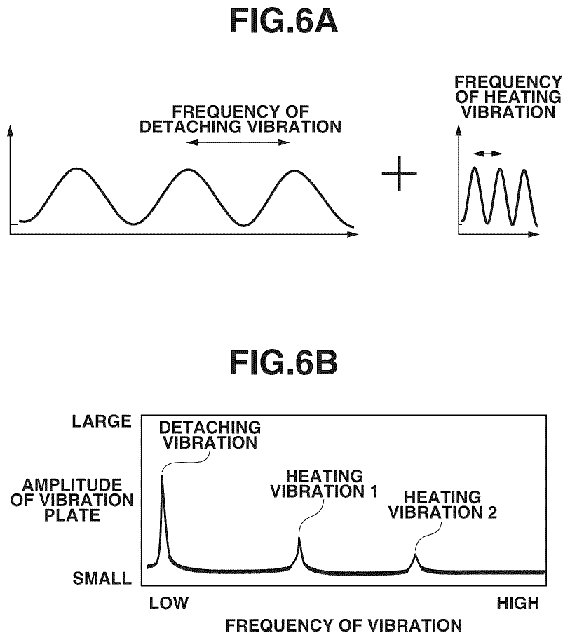

FIGS. 6A and 6B are schematic diagrams illustrating vibration modes according to the present exemplary embodiment. In the present exemplary embodiment, the vibration unit 107 is vibrated in at least two modes, i.e., a first mode for increasing a temperature of the vibration transmission material 129 and a second mode for detaching the cells from the culture container 100.

In order to efficiently execute temperature management and cell detachment, it is preferable that the vibrations generated in the first and the second modes be vibrations of an ultrasonic band. Further, it is possible to execute temperature management more promptly by making the frequency of the first mode (heating vibration) be higher than the frequency of the second mode (detaching vibration). For example, the second mode may be a vibration having a frequency swept from 20 kHz to 200 kHz. By using the swept frequency, a vibration at a resonance frequency can be generated, and a vibration having a large amplitude can be acquired. Further, for example, the first mode may be a vibration having a fixed frequency falling within a range of 100 kHz to 200 kHz. The vibration transmission material 129 can be heated more promptly by using the vibration having a comparatively high and fixed frequency.

### Process Flow of Cell Processing Method

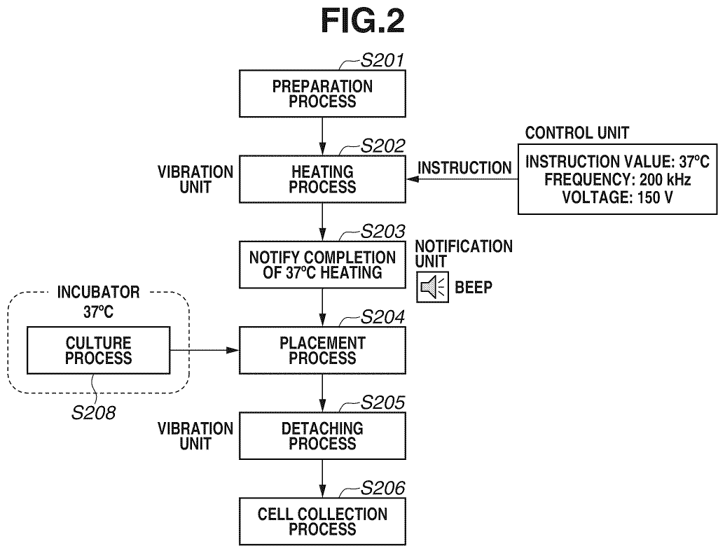

FIG. 2 is a flowchart illustrating a process flow of the cell processing method according to the present exemplary embodiment. First, in a preparation process, the user prepares the vibration unit 107 and the vibration transmission material 129 in a state where the vibration transmission material 129 is placed to make contact with the vibration unit 107 of the cell processing system (S201). In addition, the vibration transmission material 129 may be previously placed on and fixed to the vibration unit 107. The preparation of the vibration unit 107 and the vibration transmission material 129 may be automatically executed by an optional system.

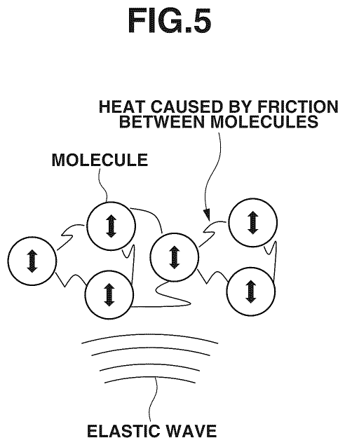

Next, the user inputs an instruction to execute preheating to the cell processing system through an input unit. According to the instruction, in a heating process (S202), a control unit of the cell processing system instructs the vibration unit 107 on a driving condition to make the vibration unit 107 start vibrating in the first mode. As illustrated in FIG. 2, in the heating process, the vibration unit 107 makes the vibration transmission material 129 vibrate by vibrating in the first mode, so that a temperature of the vibration transmission material 129 is increased. A conceptual diagram in FIG. 5 illustrates generation of heat. When the elastic wave generated by the vibration unit 107 is transmitted to the vibration transmission material 129, a temperature of the vibration transmission material 129 is increased because of frictional heat caused by friction between the molecules.

It is preferable that a temperature of the vibration transmission material 129 be set to a temperature appropriate for the type of cells as detaching targets to survive. The appropriate temperature varies depending on the cell type. However, for example, a temperature of the vibration transmission material 129 can be set to 37° C.

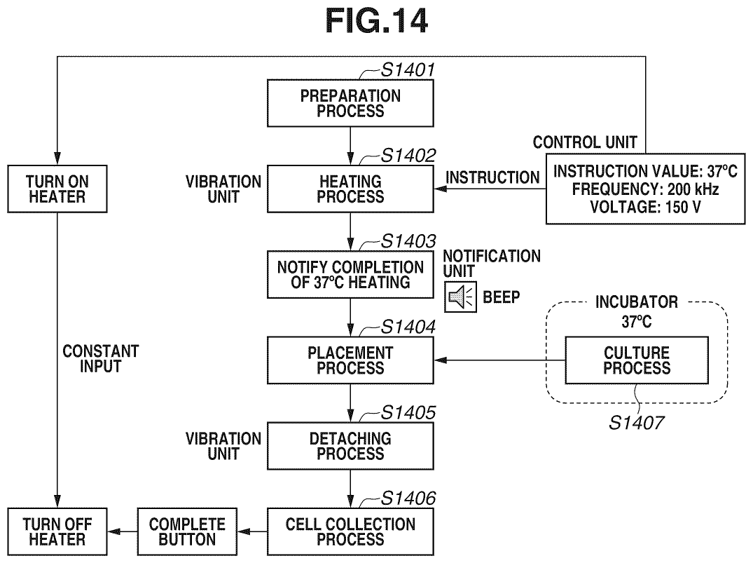

In addition, the instruction to execute preheating does not always have to be issued by the user, and the instruction may automatically be executed by the cell processing system. Further, the user may use an input unit such as a button, a touch panel, a keyboard, a mouse, or a controller. As illustrated in the flowchart in FIG. 14, a heater may be activated when the vibration unit 107 is activated according to the instruction to execute preheating if a heater is arranged on the housing 101 (S1402).

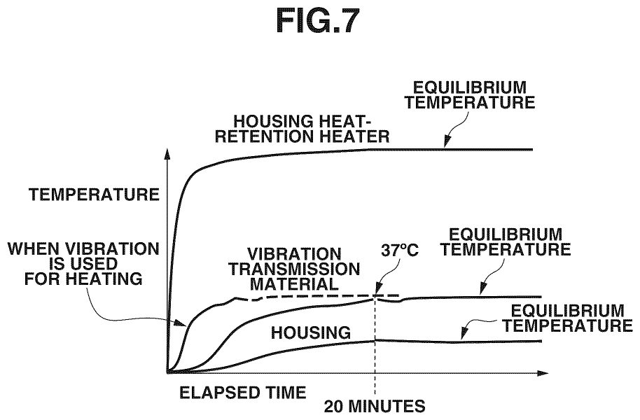

FIG. 7 is a schematic diagram illustrating a temperature change in the heating process. In a case where the heater is arranged on the housing 101, for example, a temperature of the vibration transmission material 129 is gradually increased according to an increase in the temperature of the heater after the heater is activated, although it will take some time before the temperature reaches an equilibrium temperature. Therefore, the heating process which employs the vibration in the first mode is executed in tandem with the activation of the heater. In this way, a temperature of the vibration transmission material 129 can be increased more promptly.

Herein, the cell processing system may also include a measurement unit and a notification unit. The notification unit (not illustrated) may issue a notification to the surroundings when the heating process is ended, i.e., when a temperature of the vibration transmission material 129 has reached a predetermined temperature (S203). The notification unit may issue the notification by using at least any one of light, sound, and display of characters. A temperature of the vibration transmission material 129 is measured by the measurement unit (a temperature sensor 118, for example). The notification unit issues a notification of completion of preheating, whereby the user can easily understand when to execute the placement process, resulting in being able to efficiently execute the operations.

Next, in the placement process executed after the heating process is started, the culture container 100 is placed to make contact with the vibration transmission material 129 (S204). The cells 3 held on the culture face of the culture container 100 are cultured in a culture process (S208). Through the placement process, the vibration unit 107, the vibration transmission material 129, and the culture container 100 are placed in that order. The placement process can be executed manually or automatically.

In the detaching process (S205) executed after the placement process, the control unit makes the vibration unit 107 vibrate in the second mode. In other words, the vibration unit 107 generates a vibration in the second mode in a state where the vibration transmission material 129 makes contact with the culture container 100. With this vibration, cells within the culture container 100 are vibrated and detached from the culture face of the culture container 100.

In a present exemplary embodiment, examples of the vibration mode according to the present disclosure are described.

### First Mode

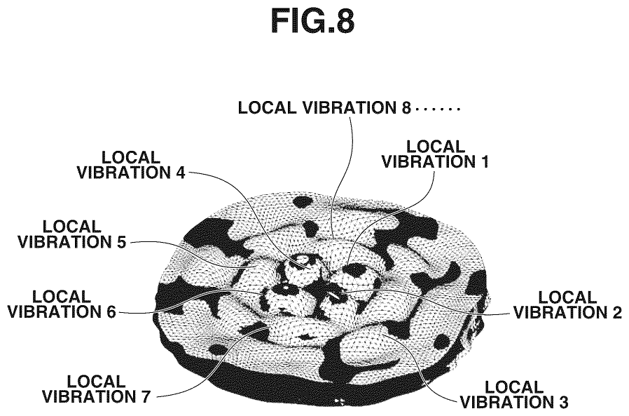

In the first mode for increasing a temperature of the vibration transmission material 129, it is preferable that a vibration have a maximum amplitude at a plurality of different places on the vibration face of the vibration unit 107 in order to promptly increase a temperature of the vibration transmission material 129. FIG. 8 is a diagram illustrating local vibrations on the vibration face (vibration plate 102) of the vibration unit 107, having a maximum amplitude at a plurality of places. When the vibration plate 102 is vibrated in a mode for generating a plurality of local vibrations, a shape of the vibration transmission material 129 which makes contact with the vibration plate 102 is significantly changed, so that a temperature of the vibration transmission material 129 can be promptly increased.

### Second Mode

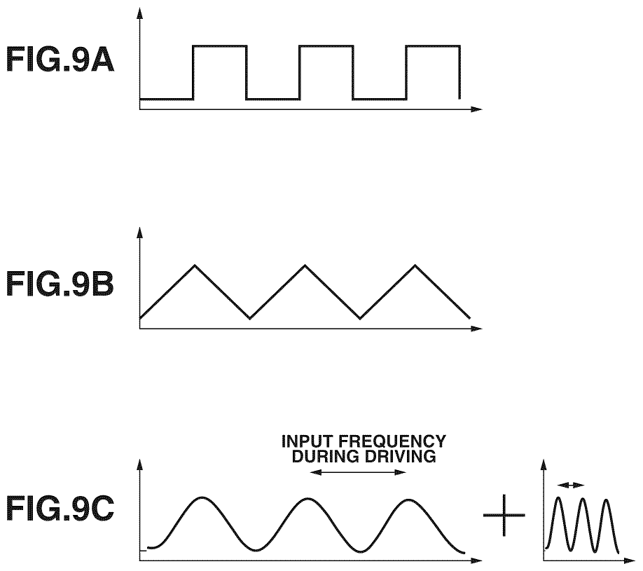

FIGS. 9A to 9C are schematic diagrams illustrating a vibration mode.

As illustrated in FIG. 9C, the first mode and the second mode may be simultaneously executed in the detaching process. In other words, the second mode executed in the detaching process may be a combination of a plurality of vibration modes. Through the second mode which includes a vibration having a frequency higher than a frequency of the vibration in the first mode, a temperature of the vibration transmission material 129 can be effectively increased. In addition, a vibration in the second mode may be a rectangular wave illustrated in FIG. 9A or a triangular wave illustrated in FIG. 9B.

A temperature of the vibration transmission material 129 can also be increased by generating a vibration in the above-described second mode after the culture container 100 is placed. Therefore, it is possible to adjust a temperature of the cells.

### Switching Between Vibration Modes

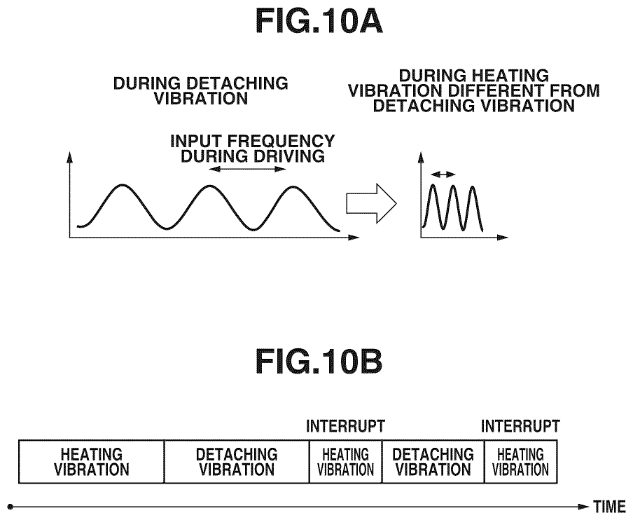

FIGS. 10A and 10B are diagrams illustrating an example of switching between the vibration modes. As illustrated in FIG. 10B, even in a case where a decrease in temperature of the vibration transmission material 129 cannot be prevented by applying only the vibration in the second vibration mode, it is possible to increase the temperature again by executing the heating process after the detaching process is started.

Further, it is preferable that the vibration frequency or the amplitude of the vibration unit 107 be changed by the control unit in a case where a temperature of the vibration transmission material 129, measured by the measurement unit (not illustrated) in the heating process, falls outside a predetermined range. By changing the vibration frequency or the amplitude, it is possible to maintain the vibration transmission material 129 at a temperature appropriate for the cells to survive. For example, in a case where a temperature of the vibration transmission material 129 is to be maintained at 37° C., it is effective to execute control for reducing the amplitude when the measured temperature is 39° C. and increasing the amplitude when the measured temperature is 35° C. Further, a target for which the measurement unit measures a temperature is not limited to the vibration transmission material 129, and the measurement unit may also measure a temperature of a part of the vibration unit 107.

### Configuration Example of Cell Processing Apparatus

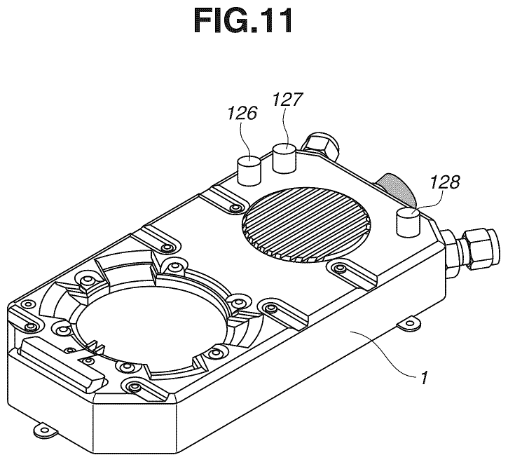

FIG. 11 is a schematic diagram illustrating a configuration example of the cell processing apparatus 1 according to a present exemplary embodiment. A housing 101 of the cell processing apparatus may include a Power button 126, a Detach button 127, and a Complete button 128. By arranging the above-described buttons, the user can use the cell processing apparatus by simply executing operations on the buttons arranged on the cell processing apparatus. Accordingly, it is possible to provide an easy-to-use space-saving cell processing apparatus.

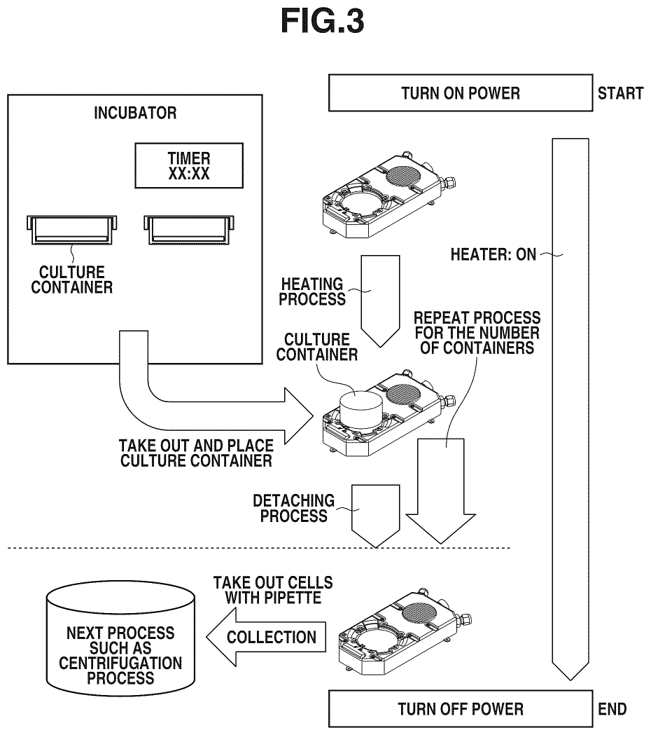

As illustrated in FIG. 3, the user operates the buttons such as the Power button 126 to use the cell processing apparatus through the following process flow. When the user presses the Power button 126, a heater for heating the housing 101 with constant input is turned on, and the vibration unit 107 starts generating a heating vibration (vibration in the first mode). Thereafter, the vibration unit 107 stops generating the heating vibration when a preset heating time has passed. At this time, a heat-retention heater is maintained in a power-on state.

Next, the user places the culture container 100 taken out from the incubator on the cell processing apparatus, and presses the Detach button 127 to start a detaching vibration (vibration in the second mode). After the user visually confirms detachment of the cells, the user presses the Complete button 128 to stop the detaching vibration.

In a case where the user continuously executes cell detachment for a plurality of culture containers 100, the user promptly places the second and subsequent culture containers 100 on the cell processing apparatus after the processing of the first culture container 100 is completed. Then, the user repeatedly executes the operations by pressing the Detach button 127 after placing the culture container 100 and by pressing the Complete button 128 after confirming detachment of the cells. When the user detaches the cells from all of the culture containers 100, the user presses the Power button 126 again to turn off the power and stops the heat-retention heater.

### Example of Temperature Adjustment Mechanism

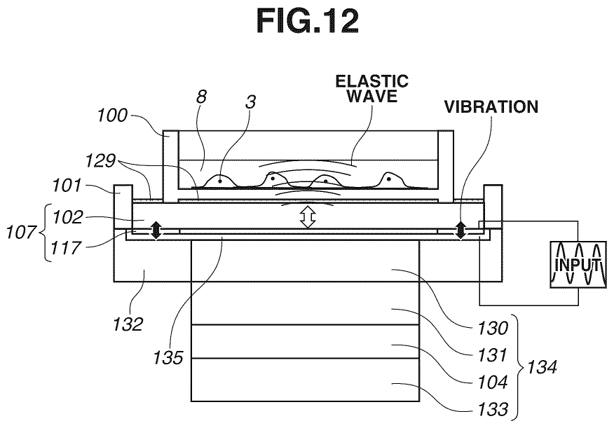

FIG. 12 is a schematic diagram illustrating an apparatus configuration of the cell processing system according to the present exemplary embodiment. In order to prevent a temperature of the cells from being increased rapidly or from being high, in some embodiments, a temperature adjustment mechanism is arranged as illustrated in FIG. 12. A cooling space 135 is arranged on the lower side of the vibration unit 107, i.e., on a side opposite to a side the culture container 100 is placed. The culture container 100 is holding the cells 3 and a culture medium 8. The cooling space 135 is covered by a wall-like heat insulation member 132 which makes contact with the housing 101. The cooling space 135 is also partitioned by a cooling apparatus 134, comprising a cooling plate 130, a cooler 131, a cooling fin 104 and a cooling fan 133. The cooling plate 130 having an excellent heat conductivity, arranged on the lower side. The cooling plate 130 abuts on the cooler 131 such as a Peltier element, and the cooling fin 104 is arranged on a side from which heat is exhausted. The cooling fan 133 for blowing the exhausted heat is arranged on the lower side of the cooling fin 104. By arranging the above-described temperature adjustment mechanism, a temperature of the cells can be managed with higher precision.

### Example of Cell Processing System Having Storage Unit

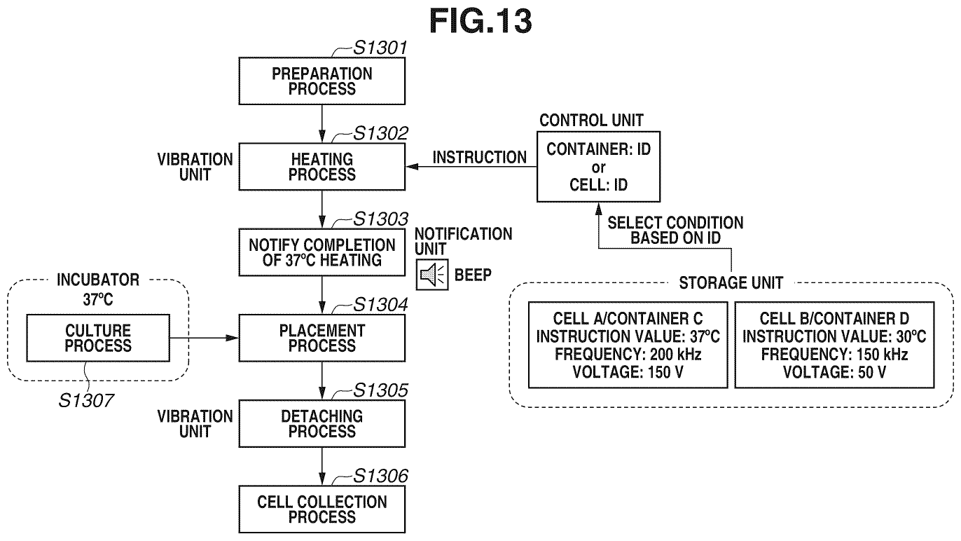

The cell processing system according to the present exemplary embodiment may have a storage unit. The storage unit can be configured of a storage medium such as an optional memory or an optical disk. FIG. 13 is a flowchart illustrating the cell processing method according to the present exemplary embodiment. The storage unit stores a processing condition associated with at least any one of a cell type, a culture condition of cells, and a type of the culture container 100.

The processing condition includes at least any one of a temperature, a vibration frequency, and a driving voltage. The control unit refers to the processing condition associated with identification (ID) information stored in the storage unit, and controls the vibration generated by the vibration unit 107 based on the identification information of the culture container 100 or the cell as a processing target (S1302). The identification information of the processing target may be optically read and acquired from a bar code, may be manually input by the user, or may be selected by the user from a list. Through the above-described configuration, it is possible to execute cell detachment according to a state different for each sample.

### Program for Implementing Cell Processing Method

Some embodiments can be realized by executing the following processing. In other words, some embodiments of the present disclosure can be realized by supplying software (program) for implementing one or more functions according to the above-described described various exemplary embodiments to a system or an apparatus via a network or a storage medium. Some embodiments of the present disclosure can also be realized through the processing in which a computer (or a CPU or a micro processing unit (MPU)) of the system or the apparatus reads and executes the program. The computer includes one or a plurality of processors or circuits, and may also include a network of a plurality of separated computers, separated processors, or circuits in order to read and execute a computer executable instruction.

In this case, the processors or the circuits may include a central processing unit (CPU), a micro processing unit (MPU), and a graphics processing unit (GPU). The processors or the circuits may also include an application specific integrated circuit (ASIC) or a field programmable gate array (FPGA). Furthermore, the processors or the circuits may include a digital signal processor (DSP), a data flow processor (DFP), or a neural processing unit (NPU).

According to the present disclosure, it is possible to provide a cell processing method capable of adjusting a temperature of a member which makes contact with a culture container to a desirable range, when cells adhering to a culture face of the culture container is to be detached therefrom. Further, according to the present disclosure, it is possible to provide a cell processing apparatus and a cell processing system capable of adjusting a temperature of a member which makes contact with a culture container to a desirable range, when cells adhering to a culture face of the culture container is to be detached therefrom.

While the present disclosure has described exemplary embodiments, it is to be understood that some embodiments are not limited to the disclosed exemplary embodiments. The scope of the following claims is to be accorded the broadest interpretation so as to encompass all such modifications and equivalent structures and functions.

This application claims priority to Japanese Patent Application No. 2023-186021, which was filed on Oct. 30, 2023 and which is hereby incorporated by reference herein in its entirety.

What is claimed is:

1. A cell processing method for detaching cells adhering to a culture face of a culture container from the culture container by using a cell processing system including a vibration unit configured to generate vibrations, the cell processing method comprising:
heating a vibration transmission material to increase a temperature of the vibration transmission material by making the vibration unit generate a vibration in a first mode in a state where the vibration unit makes contact with the vibration transmission material; and
detaching the cells from the culture container by making the vibration unit generate a vibration in a second mode different from the first mode after the heating is started, in a state where the vibration transmission material makes contact with the culture container.

2. The cell processing method according to claim 1, wherein the vibrations in the first and the second modes are vibrations of an ultrasonic band.

3. The cell processing method according to claim 1, wherein the vibration in the first mode is a vibration having a frequency higher than a frequency of the vibration in the second mode.

4. The cell processing method according to claim 1, wherein the vibration unit includes a vibrator including a piezoelectric material and a vibration plate, and generates the vibrations in the first and the second modes because of a driving voltage applied to the vibrator.

5. The cell processing method according to claim 1, wherein the vibration in the first mode is a vibration having a maximum amplitude at a plurality of different places on a vibration face of the vibration unit.

6. The cell processing method according to claim 1, wherein the cell processing method at least includes a time when the heating and the detaching are executed simultaneously.

7. The cell processing method according to claim 1, wherein the vibration unit generates the vibration in the first mode after the detaching is started.

8. The cell processing method according to claim 1, wherein the vibration in the second mode is a rectangular wave or a triangular wave.

9. The cell processing method according to claim 1, wherein a vibration frequency or an amplitude of the vibration unit is changed in a case where a temperature of the vibration transmission material falls outside a predetermined range in at least any one of the heating and the detaching.

10. The cell processing method according to claim 1, wherein the vibration transmission material is a solid or a liquid.

11. The cell processing method according to claim 10, wherein the vibration transmission material contains silicone rubber or water.

12. A non-transitory computer-readable medium storing instructions that, when executed by a computer, cause the computer to execute a cell processing method for detaching cells adhering to a culture face of a culture container from the culture container by using a cell processing system including a vibration unit configured to generate vibrations, the cell processing method comprising:
heating a vibration transmission material to increase a temperature of the vibration transmission material by making the vibration unit generate a vibration in a first mode in a state where the vibration unit makes contact with the vibration transmission material; and
detaching the cells from the culture container by making the vibration unit generate a vibration in a second mode different from the first mode after the heating is started, in a state where the vibration transmission material makes contact with the culture container.

13. A cell processing apparatus that detaches cells adhering to a culture face of a culture container from the culture container, the cell processing apparatus comprising:
a vibration unit configured to generate vibrations; and
a control unit configured to control the vibrations generated by the vibration unit,
wherein the control unit causes the vibration to be transmitted to a vibration transmission material to cause a temperature of the vibration transmission material to be increased by controlling the vibration unit to vibrate in a first mode in a state where the vibration unit makes contact with the vibration transmission material, and
wherein the control unit causes the cells to be detached from the culture container by controlling the vibration unit to vibrate in a second mode different from the first mode in a state where the vibration transmission material makes contact with the culture container.

14. The cell processing apparatus according to claim 13, wherein the vibration in the first mode is a vibration having a frequency higher than a frequency of the vibration in the second mode.

15. The cell processing apparatus according to claim 13, further comprising a storage unit configured to store a processing condition associated with at least any one of a cell type of the cells, a culture condition of the cells, and a type of the culture container,
wherein the control unit controls the vibrations generated by the vibration unit according to the processing condition.

16. The cell processing apparatus according to claim 13, further comprising:
a measurement unit configured to measure a temperature of the vibration transmission material; and
a notification unit configured to issue a notification by using at least any one of light, sound, and display of characters when the temperature measured by the measurement unit reaches a predetermined temperature.

17. A cell processing system that detaches cells adhering to a culture face of a culture container from the culture container, the cell processing system comprising:
a vibration unit configured to generate vibrations; and
a control unit configured to control the vibrations generated by the vibration unit,
wherein the control unit causes the vibration to be transmitted to a vibration transmission material to cause a temperature of the vibration transmission material to be increased by controlling the vibration unit to vibrate in a first mode in a state where the vibration unit makes contact with the vibration transmission material, and
wherein the control unit causes the cells to be detached from the culture container by controlling the vibration unit to vibrate in a second mode different from the first mode in a state where the vibration transmission material makes contact with the culture container.

18. The cell processing system according to claim 17, wherein the vibration in the first mode is a vibration having a frequency higher than a frequency of the vibration in the second mode.

19. The cell processing system according to claim 17, further comprising a storage unit configured to store a processing condition associated with at least any one of a cell type of the cells, a culture condition of the cells, and a type of the culture container,
wherein the control unit controls the vibrations generated by the vibration unit according to the processing condition.

20. The cell processing system according to claim 17, further comprising:
a measurement unit configured to measure a temperature of the vibration transmission material; and
a notification unit configured to issue a notification by using at least any one of light, sound, and display of characters when the temperature measured by the measurement unit reaches a predetermined temperature.
